(19) United States
(12) Patent Application Publication
(10) Pub. No.: US 2009/0018426 A1
(43) Pub. Date: Jan. 15, 2009

(54) DEVICE AND METHODS FOR CALIBRATING ANALYTE SENSORS

(75) Inventors: David R. Markle, Berwyn, PA (US); William Markle, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(73) Assignee: Glumetrics, Inc., Irvine, CA (US)

(21) Appl. No.: 12/118,429

(22) Filed: May 9, 2008

Related U.S. Application Data
(60) Provisional application No. 60/917,309, filed on May 10, 2007.

(51) Int. Cl. A61B 5/145

(52) U.S. Cl. 600/365; 73/1.02

(57) ABSTRACT

The present invention relates to methods and systems for multipoint calibration of an analyte sensor. More specifically, the methods can be used to calibrate glucose sensors.

### CROSS-REFERENCE TO RELATED APPLICATIONS

This application claims the priority benefit to U.S. Provisional No. 60/917,309 filed May 10, 2007, the entirety of which is hereby incorporated by reference herein.

### BACKGROUND OF THE INVENTION

1. Field of the Invention

An improved method for multipoint calibration of analyte sensors is disclosed in accordance with preferred embodiments of the present invention. In preferred embodiments, the method is adapted to calibrate sensors that monitor the concentration of sugars, i.e., glucose or fructose.

2. Description of the Related Art

Analyte sensors, such as glucose sensors, for detecting and measuring desired characteristics, such as glucose content, of liquid samples are well-known. To assure analyte measurement accuracy, an analyte sensor requires calibration. Errors due to miscalibration of analyte sensors could lead to significant errors in determining the concentration of an analyte of interest. Therefore, prior to use, it is desirable to check a sensor for a linear response to analyte concentration. This is preferably done immediately prior to use.

Thus, there is a significant need for methods that would improve the calibration of analyte sensors. It is therefore desirable to provide a quick, convenient and accurate method of calibrating of an analyte sensor.

### SUMMARY OF THE INVENTION

In preferred embodiments, the present invention concerns a method for multipoint calibration of an analyte sensor, especially an analyte sensor for determining in vivo, especially sugars, such as glucose or fructose, in physiological media.

A method for multipoint calibration of an analyte sensor is disclosed in accordance with some embodiments of the present invention. The method comprises: providing a vessel containing a first solution, wherein a sensing region of the sensor is in contact with the first solution; obtaining a first calibration signal from the sensor; adding an amount of a second solution into the vessel by means of a syringe, whereupon the sensor produces another calibration signal; and calculating a calibration factor using the first calibration signal and any additional calibration signals, thereby calibrating the analyte sensor.

A method for multipoint calibration of an analyte sensor is disclosed in accordance with another embodiment of the present invention. The method comprises: providing a vessel comprising at least two linearly adjacent chambers, wherein each chamber contains a solution, and wherein each chamber is separated from the chamber adjacent to it by a divider such that the solution in each chamber is substantially prevented from mixing with the solution in any other chamber; wherein a sensing region of the sensor is in contact with the solution in one of the chambers; obtaining a first calibration signal from the sensor; moving the sensing region of the sensor into an adjacent chamber, thereby contacting the sensing region with the solution in the adjacent chamber, whereupon the sensor produces an additional calibration signal; and calculating a calibration factor using the first calibration signal and any additional calibration signals, thereby calibrating the analyte sensor.

A method for multipoint calibration of an analyte sensor is disclosed in accordance with another embodiment of the present invention. The method comprises: exposing the sensing region of the sensor to a solution, whereupon the sensor produces a first calibration signal; combining at least one timed-release capsule with the solution, wherein the timed-release capsule contains an analyte; allowing each timed-release capsule to release the analyte contained within it, whereupon the sensor produces another calibration signal; and calculating a calibration factor using the first calibration signal and any additional calibration signals, thereby calibrating the analyte sensor.

A method for multipoint calibration of an analyte sensor is disclosed in accordance with another embodiment of the present invention. The method comprises: providing a vessel containing a solution, wherein a sensing region of the sensor is in contact with the solution; and wherein the vessel comprises at least one rupturable chamber containing an analyte, wherein the analyte is initially substantially separated from the solution; obtaining a first calibration signal from the sensor; rupturing each rupturable chamber, thereby releasing the analyte within it, whereupon the sensor produces another calibration signal; and calculating a calibration factor using the first calibration signal and any additional calibration signals, thereby calibrating the analyte sensor.

A kit for multipoint calibration of an analyte sensor is disclosed in accordance with another embodiment of the present invention. The kit includes a vessel containing a calibration solution, the vessel having a port for a sensor to access the calibration solution. The kit according to this embodiment of the present invention further includes a syringe for delivery of an analyte.

A ready-to-calibrate and deploy, sterilized analyte sensor kit is disclosed in accordance with another embodiment of the present invention. The kit comprises: an analyte sensor comprising an elongate body having an indicator system disposed along a distal portion of the elongate body; a calibration vessel comprising a sensor port through which the distal portion of the sensor is sealably retained within the vessel until retracted for use, and the vessel further comprising a calibration means in fluid communication with the vessel, wherein the sensor and vessel are pre-assembled, sterilized and sealed within a sterile package, ready for calibration and deployment.

In one variation to the above-described kit, the calibration means comprises a calibration port in fluid communication with the vessel and a syringe comprising a calibration solution fluidly-coupled to the vessel via the calibration port.

A ready-to-calibrate and deploy, sterilized analyte sensor kit is disclosed in accordance with another embodiment. The kit comprises: an analyte sensor comprising an elongate body having an indicator system disposed along a distal portion of the elongate body and an coupling member configured to interface with an analyte monitor comprising a calibration algorithm; a calibration apparatus comprising a calibration chamber sized to slidably receive and accommodate therein the distal portion of the elongate body of the sensor, an adjustable sealing means for sealing the distal portion within the calibration chamber, an infusion port fluidly coupled to the calibration chamber, and a fluid waste receptacle fluidly coupled to the calibration chamber; and wherein the analyte sensor is slidably engaged within the calibration apparatus, sterilized and sealed within a sterile package, ready for calibration and deployment.

A method of calibrating an analyte sensor using the above kit is also disclosed. The method comprises: providing the above analyte sensor kit; providing at least first and second calibration solutions in separate syringes; providing the analyte monitor; coupling the analyte sensor to the analyte monitor via the coupling member and initiating the calibration algorithm; infusing the first calibration solution into the calibration chamber; allowing the sensor to equilibrate; infusing the second calibration solution into the calibration chamber, collecting displaced fluid in the waste receptacle; and allowing the sensor to equilibrate, wherein the calibration algorithm automatically calibrates the sensor.

### BRIEF DESCRIPTION OF THE DRAWINGS

FIG. 1 depicts a system for multipoint calibration of an analyte sensor comprising a vessel and a syringe.

FIG. 2 depicts a system for multipoint calibration of an analyte sensor comprising a vessel comprising three chambers.

FIGS. 3A and 3B depict various configurations of a timed-release capsule for use in multipoint calibration of an analyte sensor. The timed-release capsules comprise a membrane and an analyte.

FIG. 4 depicts a system for multipoint calibration of an analyte sensor comprising a vessel with rupturable chambers.

FIG. 5 depicts a system for multipoint calibration of an analyte sensor comprising a vessel and a valve.

FIG. 6 depicts another calibration apparatus in accordance with an embodiment of the invention.

FIG. 7 depicts another calibration apparatus in accordance with another embodiment of the invention.

FIG. 8 yet another calibration apparatus in accordance with another embodiment of the invention.

FIG. 9 shows a calibration apparatus with a vent in accordance with a preferred embodiment of the invention.

Throughout the figures, the same reference numerals and characters, unless otherwise stated, are used to denote like features, elements, components or portions of the illustrated embodiments. Moreover, while the subject matter of this application will now be described in detail with reference to the figures, it is done so in connection with the illustrative embodiments. It is intended that changes and modifications can be made to the described embodiments without departing from the true scope and spirit of the subject invention as defined in part by the appended claims.

### DETAILED DESCRIPTION OF THE PREFERRED EMBODIMENT

Methods and systems for multipoint calibration of an analyte sensor are disclosed in accordance with preferred embodiments of the present invention. Prior to use of an analyte sensor, to ensure accuracy, it is desirable to check the sensor for a linear response to analyte concentration using the calibration methods disclosed herein. This is preferably done immediately prior to use. Various embodiments of apparatuses and procedures described herein will be discussed in terms of glucose sensors. For example, WO 2008/001091A1 describes some solutions to the problem of sensor calibration while maintaining sterility and is incorporated herein in its entirety by reference thereto. However, many aspects of the present invention may find use in other types of analyte sensors.

### DEFINITIONS

In order to facilitate an understanding of the disclosed invention, a number of terms are defined below.

The term “calibration” as used herein is a broad term, and is to be given its ordinary and customary meaning to a person of ordinary skill in the art (and it is not to be limited to a special or customized meaning), and refers without limitation to the relationship and/or the process of determining the relationship between the sensor data and corresponding reference data, which may be used to convert sensor data into meaningful values substantially equivalent to the reference.

The term “multipoint calibration” as used herein is a broad term, and is to be given its ordinary and customary meaning to a person of ordinary skill in the art (and it is not to be limited to a special or customized meaning), and refers without limitation to calibration, as defined above, wherein more than one data point is used.

The term “sensor” or “analyte sensor” encompasses any device that can be used to measure the concentration of an analyte, or derivative thereof, of interest. Sensors can be, for example, electrochemical, chemical piezoelectric, thermoelectric, acoustic, or optical. Preferred sensors for detecting blood analytes generally include electrochemical devices and chemical devices. Examples of electrochemical devices include (list examples of such devices).

The term “sensing region” as used herein is a broad term, and is to be given its ordinary and customary meaning to a person of ordinary skill in the art (and it is not to be limited to a special or customized meaning), and refers without limitation to the region of a monitoring device or sensor responsible for the detection of a particular analyte.

The term “vessel” as used herein is a broad term, and is to be given its ordinary and customary meaning to a person of ordinary skill in the art (and it is not to be limited to a special or customized meaning), and refers without limitation to a hollow utensil used as a container, especially for liquids. Examples of vessels suitable for use with the present invention include, but are not limited to, containers, tubes, tubular bodies, tonometers, capsules, tubes, vials, capillary collection devices, and cannulas. In some embodiments, the vessel is a tonometer. In another embodiment, the vessel is a hollow, enclosed tube.

The term “analyte” is used herein to denote any physiological analyte of interest that is a specific substance or component that is being detected and/or measured in a chemical, physical, enzymatic, or optical analysis. A detectable signal (e.g., a chemical signal or electrochemical signal) can be obtained, either directly or indirectly, from such an analyte or derivatives thereof. Furthermore, the terms “analyte” and “substance” are used interchangeably herein, and are intended to have the same meaning, and thus encompass any substance of interest. In preferred embodiments, the analyte is a physiological analyte of interest, for example, glucose, or a chemical that has a physiological action, for example, a drug or pharmacological agent.

Analytes may include naturally occurring substances, artificial substances, metabolites, and/or reaction products. In some embodiments, the analyte for measurement by the sensors and methods disclosed herein is glucose. However, other analytes are contemplated as well.

Although the term “glucose” is used herein below, it is to be understood most polyhydroxyl-containing organic compounds (carbohydrates, 1,2-diols, 1,3-diols and the like) in a solution may used for multipoint calibration of the glucose sensor.

The term “port” as used herein is a broad term, and is to be given its ordinary and customary meaning to a person of ordinary skill in the art (and it is not to be limited to a special or customized meaning), and refers without limitation to an opening or aperture, for example, in the side of a vessel.

The term “substantially” as used herein is a broad term, and is to be given its ordinary and customary meaning to a person of ordinary skill in the art (and it is not to be limited to a special or customized meaning), and refers without limitation to a sufficient amount that provides a desired function.

The term “comprising” as used herein is synonymous with “including,” “containing,” or “characterized by,” and is inclusive or open-ended and does not exclude additional, unrecited elements or method steps.

As used herein, the term “proximal,” as is traditional, refers to the end portion of the apparatus that is closest to the operator, while the term “distal” refers to the end portion that is farthest from the operator.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

### Description of Embodiments

The systems and methods described herein are in connection with multipoint calibration, and in particular with the calibration of a glucose sensor, as further discussed below. In some embodiments, the methods can be used to, calibrate an analyte sensor for monitoring the concentration of a sugar in vitro. In other embodiments, the methods can be used to calibrate an analyte sensor for monitoring the concentration of a sugar in physiological media. In another embodiment, the methods can be used to calibrate an analyte sensor for monitoring in vivo, the concentration of sugars such as glucose or fructose, in physiological media. In another embodiment, the methods can be used to calibrate sensors that monitor the concentration of sugars, i.e., glucose or fructose, in blood while implanted intravascularly. In another embodiment, the analyte sensor is a pH sensor.

In preferred embodiments, the analyte sensor is a glucose sensor. As known to those skilled in the art, there are a variety of sensors used for monitoring the concentration of glucose in a fluid. The sensor(s) to be calibrated by the disclosed methods may be, for example, electrochemical, piezoelectric, thermoelectric, acoustic, or optical. Non-limiting examples of analyte sensors may be found with reference to co-pending applications U.S. application Ser. Nos. 11/671,880, filed on Feb. 6, 2007, entitled “OPTICAL DETERMINATION OF PH AND GLUCOSE”; 60/888,477, filed on Feb. 6, 2007, entitled “OPTICAL SYSTEMS AND METHODS FOR RATIOMETRIC MEASUREMENT OF BLOOD GLUCOSE CONCENTRATION”; and Ser. No. 11/296,898, filed on Dec. 7, 2005, entitled “OPTICAL DETERMINATION OF GLUCOSE USING BORONIC ACID ADDUCTS”; the entire disclosures of which are incorporated herein by reference thereto. In some embodiments, the analyte sensor is an intravascular glucose sensor.

A glucose solution suitable for use in the present invention may have a concentration of glucose, for example, between 0 mg/dL and 2 g/dL, and more preferably between about 0 to 500 mg/dL. In some embodiments, the glucose solution further comprises phosphate buffered saline (PBS), which is comprised of a phosphate buffer and sodium chloride. The PBS is used to balance the osmolarity of the glucose solution to a physiological osmolarity level and can be used to adjust the pH to between 6 to 8.

The calibration methods disclosed can be used with any calculation method useful for determining a calibration factor. The calculation of the calibration factor can be obtained, for example, using linear regression, least squares linear regression, non-linear regression, or a non-linear regression technique.

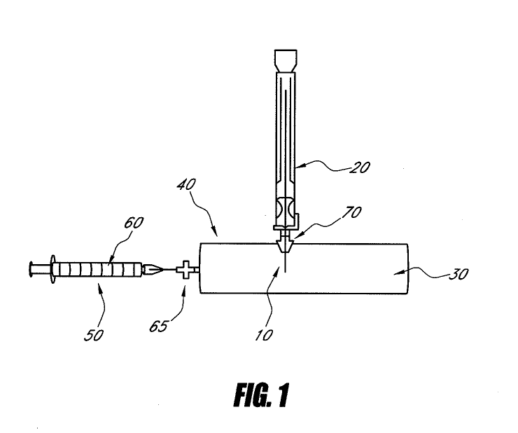

FIG. 1 shows some embodiments of a system that can be used to perform a variety of methods or procedures. In some embodiments, as discussed more fully below, the sensing region 10 of the analyte sensor 20 is in contact with a first solution 30 in a vessel 40. A first calibration signal is produced by the sensor when the sensing region is exposed to the first solution. In the illustrated embodiment, a syringe 50 is used to add a second solution 60 to the vessel. In some embodiments, the syringe is inserted through a first port 65. In the illustrated embodiment, the second solution contains analyte, depicted as dots inside the syringe and in the calibrating solution. The sensor produces another calibration signal as a result of the change in analyte concentration of the solution in the vessel. The calibration signals are used to calculate a calibration factor, thereby calibrating the analyte sensor.

In some embodiments, the first solution does not contain glucose. The first solution can be, for example, water or PBS with a pH between 6 to 8. In another embodiment, the first solution is a glucose solution. In some embodiments, the second solution is a glucose solution. In another embodiment, the second solution does not contain glucose. The concentration of glucose in the first and second solutions should differ from each other. For example, in embodiments where the first solution does not contain glucose, it is desirable for the second solution to contain glucose. The addition of the second solution to the first solution changes the glucose concentration of the solution in contact with the sensor. The sensor produces a calibration signal in response to the new glucose concentration.

In embodiments where the analyte sensor is a pH sensor, the second solution may be an acid. Alternatively, the second solution may be a base.

The syringe used to add the second solution can have stops for adding a premeasured amount of the second solution. The stops allow an operator to conveniently add a known quantity of the second solution to the vessel. For example, the syringe may have stops for delivering 1 ml increments of the second solution. The syringe can have any number of stops, for example, from one stop to ten stops. Preferably, the syringe has three stops. In some embodiments, the syringe is pre-filled with the second solution. In another embodiment, the operator fills the syringe with the second solution immediately prior to calibration.

In some embodiments, the second solution is added to the solution in the vessel two times. After each addition, a calibration signal is produced by the sensor, and the calibration factor is calculated using the first calibration signal and the two additional calibration signals. One to four data points, and preferably two to three data points, can be used for calibration of the sensor.

In some embodiments, the sensing region of the sensor is inserted through a port 70 of a vessel 40, thereby contacting the first solution in the vessel.

In some embodiments, additional syringes containing additional solutions may be used to vary the concentration of analyte inside the vessel. In some embodiments, the additional syringes are inserted through the first port 65. The first port 65 may be adapted to accept any number of syringes.

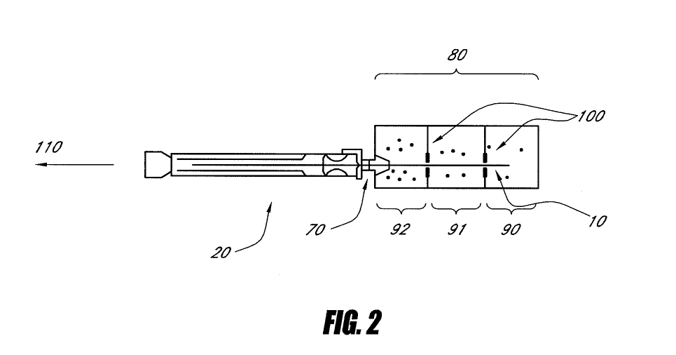

FIG. 2 shows another embodiment of a system that can be used to perform a variety of methods or procedures. In some embodiments, described more fully below, the vessel 80 has at least two (the illustrated embodiment depicts three) linearly adjacent chambers 90, 91, and 92. Each chamber contains a solution. The chambers are separated from the one another by a divider 100, which substantially prevents the solution in each chamber from mixing with the solution in any other chamber. In the illustrated embodiment, the sensing region 10 of the sensor 20 is in contact with the solution in the most distal chamber 90. Before the sensor is moved, a first calibration signal is obtained. The sensing region of the sensor is then moved 110 into the adjacent chamber 91, whereupon the sensor produces a second calibration signal. In the illustrated embodiment, the sensing region of the sensor is then retracted into the most proximal chamber 92, whereupon the sensor produces a third calibration signal. A calibration factor is calculated using the calibration signals, thereby calibrating the analyte sensor.

In some embodiments, the step of moving the sensing region is carried out by retracting the sensor into an adjacent chamber. In another embodiment, the step of moving the sensing region is carried out by advancing the sensor into an adjacent chamber. The step of moving the sensing region into an adjacent chamber can be repeated any number of times. In some embodiments, the step of moving the sensing region is carried out at least twice. In another embodiment, the step of moving the sensing region is carried out three times.

The vessel may comprise any number of chambers greater than one. In some embodiments, the vessel comprises three chambers. In another embodiment, the vessel comprises four chambers.

The solution in each chamber may or may not contain an analyte. In some embodiments, the solution in the chamber is a glucose solution. In embodiments where the solution is a glucose solution, the analyte is glucose. In some embodiments, the solution in each chamber has a glucose concentration of, for example, between 0 mg/dL and 2 g/dL, and more preferably between about 0 to 500 mg/dL. In some embodiments, the glucose solution further comprises phosphate buffered saline (PBS), which is comprised of a phosphate buffer and sodium chloride. The PBS is used to balance the osmolarity of the glucose solution to a physiological osmolarity level and can be used to adjust the pH to between 6 to 8.

Preferably, the concentration of analyte in the solution in each chamber differs from the concentration of analyte in the solution in any other chamber. In some embodiments, the vessel comprises three chambers: a first chamber, a middle chamber, and a last chamber, wherein each chamber contains a solution having a different analyte concentration. In some embodiments, the analyte concentration of the solution increases as the sensing region is moved proximally. In some embodiments, the first chamber does not contain analyte. In some embodiments, the analyte concentration of the solution in the first chamber is 400 mg/dL, the analyte concentration of the solution in the middle chamber is 100 mg/dL, and the analyte concentration of the solution in the last chamber is 0 mg/dL. In another embodiment, the glucose concentration of the solution in the first chamber is 0 mg/dL, the glucose concentration of the solution in the middle chamber is 400 mg/dL, and the glucose concentration of the solution in the last chamber is 100 mg/dL.

In some embodiments, the sensing region 10 of the analyte sensor 20 is inserted through the port 70 of a vessel 80.

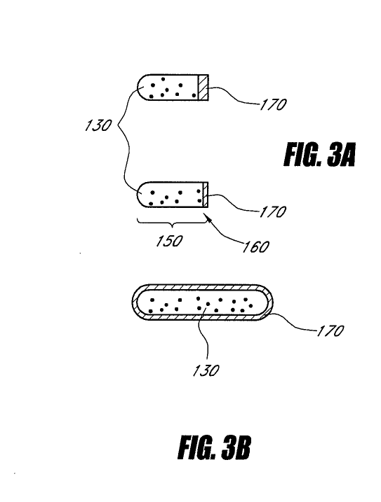

FIGS. 3A and 3B depict various configurations of a timed-release capsule that can be used in another embodiment of a system that can be used to perform a variety of methods or procedures. In some embodiments, as discussed more fully below, the sensing region of a sensor is exposed to a solution, whereupon the sensor produces a first calibration signal. At least one timed-release capsule, described more fully below, is combined with the solution. The timed-release capsule contains an analyte 130. As each timed-release capsule releases the analyte contained within it into the solution, the sensor produces another calibration signal. A calibration factor is calculated using the calibration signals, thereby calibrating the analyte sensor.

In some embodiments, the analyte sensor is a glucose sensor. In an embodiment wherein a glucose sensor is being calibrated the analyte contained in the timed-release capsule is glucose. The glucose exists at a concentration of, for example, between 0 mg/dL and 2 g/dL, and more preferably between about 0 to 500 mg/dL.

The solution may be any suitable for calibrating the analyte sensor. The solution may be, for example, comprised of a phosphate buffer or PBS.

A timed-release capsule suitable for use in the present invention can be, for example, a capsule containing a reservoir of analyte and having a degradable membrane or barrier that can dissolve in a solvent, as discussed more fully below. Such a solvent can be, for example, water. The capsule can have a variety of configurations, including the configurations depicted in FIGS. 3A and 3B. The capsule can comprise, for example, a tube-like structure 150 comprising an opening 160, wherein a degradable membrane or barrier 170 seals the opening. In another embodiment, the membrane or barrier can form the entire capsule itself, and once dissolved, would release the analyte. Examples of degradable polymers include, but are not limited to, polylactic acid, polyglycolic acid, polylactic-co-glycolic acid and polyanhydrides.

The timed-release capsule can take any amount of time to release the analyte contained within it. The timed-release can take, for example, between 10 seconds and 60 minutes to release the analyte contained within it.

The timed-release capsule may comprise a degradable membrane 170. In some embodiments, the dissolution of the degradable membrane is initiated when the timed-release capsule is combined with the calibration solution. In some embodiments, the degradable membrane has a dissolution rate proportional to the thickness of the membrane. Thus, in some embodiments, the time it takes for the analyte to be released is controlled by the thickness of the membrane/barrier. The thicker the membrane or barrier, the longer it takes the membrane or barrier to degrade, and the longer it takes the analyte to be released. Where more than one timed-release capsule is combined with the solution, the timed-release capsules may have different dissolution rates. Alternatively, the timed-release capsules may have the same dissolution rate.

At least one timed-release capsule is combined with the solution, and preferably at least two timed-release capsules are combined with the solution. In some embodiments, the method comprises three timed-release capsules. In other embodiments, the method comprises one to four timed-release capsules, and more preferably two to three timed-release capsules. In embodiments where more than one timed-release capsule is combined with the calibration solution, each timed-release capsule can take either a different or the same amount of time as the other timed-release capsule(s) to release the analyte contained within it. Preferably, the timed-release capsules have different release times. The timed-release capsules can be combined with the calibration solution simultaneously, or at different times. When multiple timed-release capsules are simultaneously combined with the calibration solution and each has a distinct and known time to release, the change in the analyte concentration over time can be predicted. Multiple calibration points can thus be generated at known time intervals.

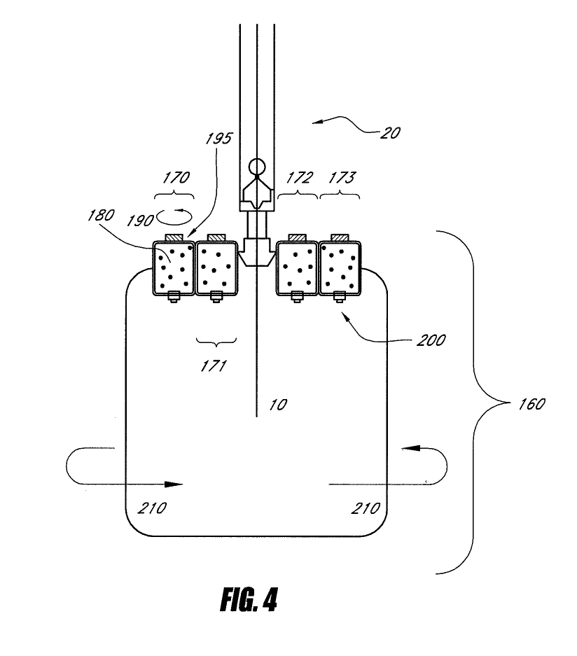

FIG. 4 shows another embodiment of a system that can be used to perform a variety of methods or procedures. In some embodiments, a vessel 160 contains a solution. The vessel further comprises at least one (the illustrated embodiment depicts four) rupturable chamber 170, 171, 172, and 173. Each rupturable chamber contains an analyte 180. The analyte is initially substantially separate from the solution. The sensing region 10 of the sensor 20 is in contact with the solution in the vessel, and a first calibration signal is obtained from the sensor. Each rupturable chamber is then ruptured, thereby releasing the analyte within. Upon release of the analyte from a rupturable chamber, the sensor produces another calibration signal. A calibration factor is calculated using the calibration signals, thereby calibrating the analyte sensor.

In some embodiments, the analyte sensor is a glucose sensor. The glucose sensor may be, for example, an intravascular glucose sensor. Preferably, the analyte is glucose. The glucose in the rupturable chamber may exist at a concentration of, for example, between 0 mg/dL and 2 g/dL, and preferably between 0 to 500 mg/dL.

The solution contained in the vessel may be any solution suitable for calibrating the analyte sensor. The solution may be, for example, comprised of phosphate buffer or PBS.

The rupturable chamber can exist in a variety of configurations. A rupturable chamber suitable for use in the present invention can be, for example, a rotatable chamber. Such a rotatable chamber may be ruptured by rotating 190 the rupturable chamber, thereby releasing the analyte. The rotatable chamber may comprise a knob 195 which an operator can grasp and twist, thereby rotating the chamber.

Rotation of the rupturable chamber may rupture the chamber by, for example, shearing. Alternatively, the rupturable chamber may, for example, comprise a valve 200, wherein the valve remains in a closed position until the rupturable chamber is rotated, whereupon the valve opens, thereby releasing the analyte. In another embodiment, the rupturable chamber is ruptured by exerting pressure on the rupturable chamber, thereby rupturing the chamber and releasing the analyte. In another embodiment, the vessel is rotated 210, thereby rotating the rupturable chamber(s) and releasing the analyte within.

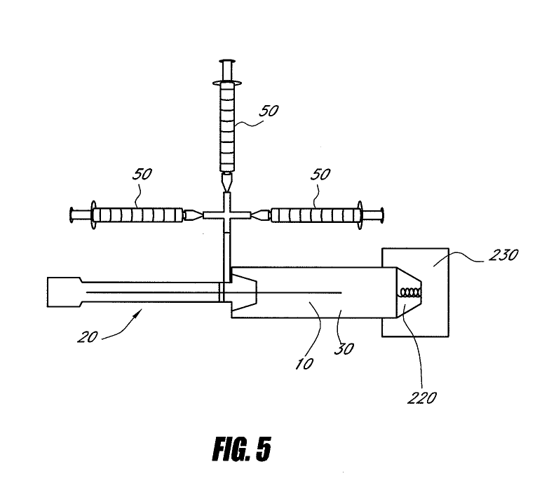

In some embodiments, it is desirable to sterilize an analyte sensor. In some embodiments, it is desirable to sterilize an analyte sensor in conjunction with a calibration system. The calibration systems described may be sterilized by a variety of methods. Once sterilized, calibration of the analyte sensor can be carried out under sterile conditions, and the calibration system may be kept sterile indefinitely. The analyte sensor maybe sterilized by, for example, autoclaving or ethylene oxide. FIG. 5 shows an embodiment of a system that can be used to perform a variety of methods or procedures. In some embodiments, a vessel 40 used for calibrating an analyte sensor comprises a valve 220 for regulating the pressure within the vessel. Such a valve allows autoclaving by maintaining the pressure such that the solution 30 does not escape from the vessel. In some embodiments, the valve comprises a spring. In some embodiments, a container 230 is used to collect any solution which may leak from the vessel during sterilization. In some embodiments, the analyte sensor in conjunction with the calibration system is placed in a bag for autoclaving.

In other embodiments, the valve 220 may be disengaged. Disengagement of the valve may be used, for example, during ethylene oxide sterilization. During ethylene oxide sterilization, the ethylene oxide gas requires access to the sensor. Disengagement of the valve permits the ethylene oxide gas to gain access to the sensor and sterilize the sensor surfaces.

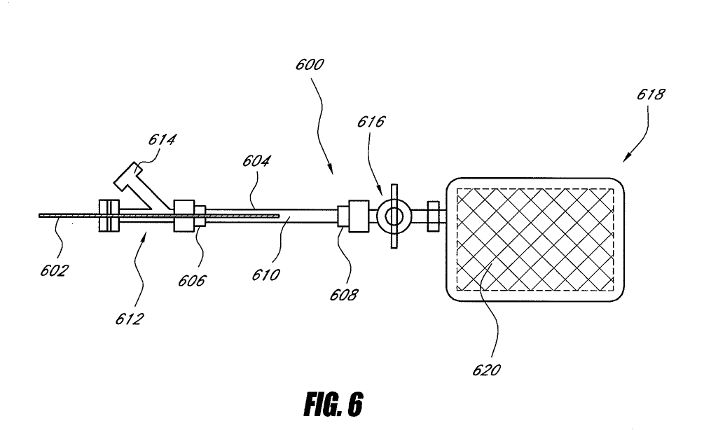

FIG. 6 shows another embodiment of a sensor calibration system 600 for calibrating a sensor 602, such as a glucose sensor. The system 600 comprises a sensor 602 disposed in a calibration chamber 604 with a proximal end 606 and a distal end 608 and a lumen 610 extending therethrough. A valve 612 is attached to the proximal end 606 of the sensor calibration chamber 604. The valve 612 also has a side port 614. In some embodiments, one end of a stopcock 616 is attached to the distal end 608 of the sensor calibration chamber 604 and the other end of the stopcock 616 is attached to a bag 618 enclosing an absorption sponge 620.

In some embodiments, the valve 612 is a Touhy-Borst valve that provides a seal around the sensor 602 and clamps the sensor 602 in place. A first calibration solution can be introduced into the system 600 via the side port 614. After a measurement has been taken, the calibration solution can be drained into the bag 618 by actuating the stopcock 616 from a closed position to an open position. The absorption sponge 620 in the bag 618 facilitates drainage of the calibration solution from the sensor calibration chamber 604. After the calibration solution is drained, the stopcock 616 can closed and a second calibration solution can be introduced. Additional calibration solutions can be introduced by draining the solution into the bag 618 before introduction of the next solution. Alternatively, in some embodiments, the introduction of the next calibration solution is used to push the previous calibration solution into the bag 618. In these embodiments, the stopcock 616 is open during the introduction of the next calibration solution.

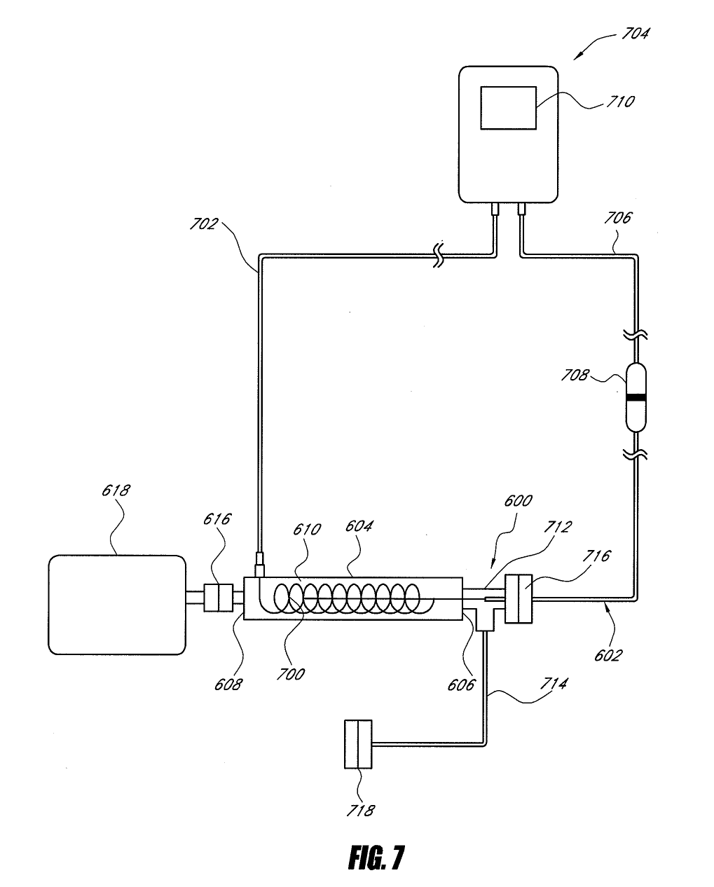

FIG. 7 shows another embodiment of a sensor calibration system 600 for calibrating a sensor 602, such as a peripheral venous glucose sensor. The system 600 comprises a sensor 602 disposed in a sensor calibration chamber 604 with a proximal end 606 and a distal end 608 and a lumen 610 extending therethrough. The sensor 602 can comprise an elongate body with a distal portion comprising analyte sensing chemistry. In some embodiments, a valve 616, such as a one-way valve like, for example, a check valve, is attached to the distal end 608 of the sensor calibration chamber 604 and the other end of the valve 616 is attached to a bag 618 for receiving calibration solution. In some embodiments, the bag 618 encloses an absorption sponge 620 (not shown).

The calibration chamber 604 has a heater 700 for heating the calibration solution before calibration measurements are taken. The calibration solution can be heated to approximately the body temperature of the patient or test subject, i.e., 37 degrees Celsius for a human patient. In some embodiments, the calibration solution can be heated to a temperature that is lower or higher than 37 degrees Celsius. For example, if the patient's body temperature is less than 37 degrees Celsius, the calibration solution can be heated to match the patient's body temperature. In addition, if the patient's peripheral body temperature is lower than the patient's core body temperature and the glucose measure will be taken at the peripheral location, the calibration solution can be heated to match the patient's lower peripheral body temperature. Alternatively, if the patient has a body temperature that is greater than 37 degrees Celsius, for example as a result of an infection, the calibration solution can be heated to a temperature greater than 37 degrees Celsius to match the patient's body temperature.

The heater 700 can comprise a resistive heating element that is coiled around or within the calibration chamber 604. In some embodiments the heater 700 and heating element may be separate from the calibration chamber 604 and can be brought into contact with the calibration chamber 604 when heating of the calibration chamber 604 is required. Separating the heater 700 from the calibration chamber 604 allows the heater 700 to be reused. In some embodiment, the heater 700 is wrapped around the calibration chamber 604. In other embodiments, the calibration chamber 604 is inserted into the heater 700. In some embodiments, the heater 700 extends along a substantial portion of the calibration chamber 604, thereby facilitating rapid and uniform heating of the calibration fluid.

In some embodiments, the heater 700 can be powered via a power line 702 that can be connected to a glucose monitor 704, which can also be connected to the glucose sensor 602 via a glucose sensor line 706 and a glucose sensor connection interface 708. Although the glucose monitor 704 and glucose sensor line 706 can be considered a part of the glucose calibration system 600, in some embodiments, the glucose monitor 704 and glucose sensor line 706 are separate from the glucose calibration system 600. In some embodiments, the glucose monitor 704 comprises a heater controller for controlling the temperature and heating rate of the heater 700, and the user can select a temperature and initiate heating using the glucose monitor 704. The power line 702 can also connect the heater controller with the heater 700. In other embodiments, the heater 700 can comprise a heater controller such that a user can directly select a temperature and initiating heating on the heater itself. In some embodiments where the heater 700 comprises a heater controller, the heater controller can be connected to the glucose monitor 704 such that the glucose monitor 704 can provide basic instructions to the heater controller, such as on/off instructions and the desired temperature. In some embodiments, the heater 700 can be supplied with power from a source independent of the glucose monitor 704. For example, in some embodiments, the heater 700 can be connected to a battery or plugged into a conventional wall socket.

Pre-heating the glucose calibration fluid can be important when the glucose sensing technology is temperature sensitive or temperature dependent. By calibrating the glucose sensor 602 at, for example, 37 degrees Celsius to match the patient's body temperature, the accuracy of in-vivo glucose measurements can be improved. The glucose monitor 704 can have a display 710 for displaying instructions to the user for performing the calibration procedure. In addition, the display 710 can display the status of the calibration procedure, including the time to complete each step, the time remaining for each step, and the results of each step. For example, the display 710 can show the temperature of the calibration fluid and can show the results of each of the glucose measurements.

The temperature of the calibration solution can be monitored by a temperature sensor, such as a thermocouple, thermistor, resistance temperature detector, or any other suitable temperature sensor. The temperature sensor can be part of or included with the glucose sensor (not shown), or the temperature sensor can be separate from the glucose sensor and reside in or on the calibration chamber 604 with the heater 700. In either case, the temperature sensor can be powered by and send data to the glucose monitor 704 via the power line 702 or the glucose sensor line 706 or via an independent power line. In other embodiments, the temperature sensor can be in communication with and powered by the heater 700 and/or heater controller.

The proximal end 606 of the calibration chamber 604 can be attached to a 3-way connector 712 that is also attached to a fill line 714 and a valve 716, which can be, for example, a Touhy-Borst valve. The fill line 714 can terminate in an infusion port 718. The glucose sensor 602 can be introduced into the calibration chamber 604 via the valve 716. Calibration solution can be introduced into the calibration chamber 604 via the infusion port 718 of the fill line 714 using, for example, a syringe with or without a hypodermic needle. In some embodiments, the location of the fill line 714 and bag 618 can be switched. If the location of the fill line 714 and bag 618 are switched, the one-way valve 616 generally remains attached to the bag 618.

To calibrate the glucose sensor 602, calibration solution with a known glucose concentration is introduced into the calibration chamber 604 via the infusion port 718 of the fill line 714. The glucose sensor 602 is introduced into the calibration chamber 604 via the valve 716 attached to the 3-way connector 712. The glucose sensor 602 can be introduced into the calibration chamber 604 either before or after the calibration solution is introduced into the calibration chamber 604. The power line 702 and glucose sensor 602 are attached to the glucose monitor 704 and this step can be done either before or after the calibration fluid is introduced into the calibration chamber 604. The calibration solution is heated by the heater 700 to about the patient's body temperature, which generally is about 37 degrees Celsius. Once the calibration solution is heated to the target temperature, a first calibration measurement can be taken. If a second calibration measurement is desired, the first calibration solution can be drained and/or flushed into the bag 618 using, for example, a second calibration solution, which has a different glucose concentration than the first calibration solution. Sufficient second calibration solution can be used to flush the first solution to ensure that substantially all of the first calibration fluid is flushed into the bag 618. Once the second solution has replaced the first solution in the calibration chamber 604, the heater 700 can be used to heat the second solution to the patient's body temperature. Once the second solution is heated to the target temperature, a second calibration measurement can be taken. If additional calibration measurements are desired, for example a third calibration measurement, the steps of draining and/or flushing the previous calibration solution with the next calibration solution and then heating the next calibration solution before taking the calibration measurement can be repeated.

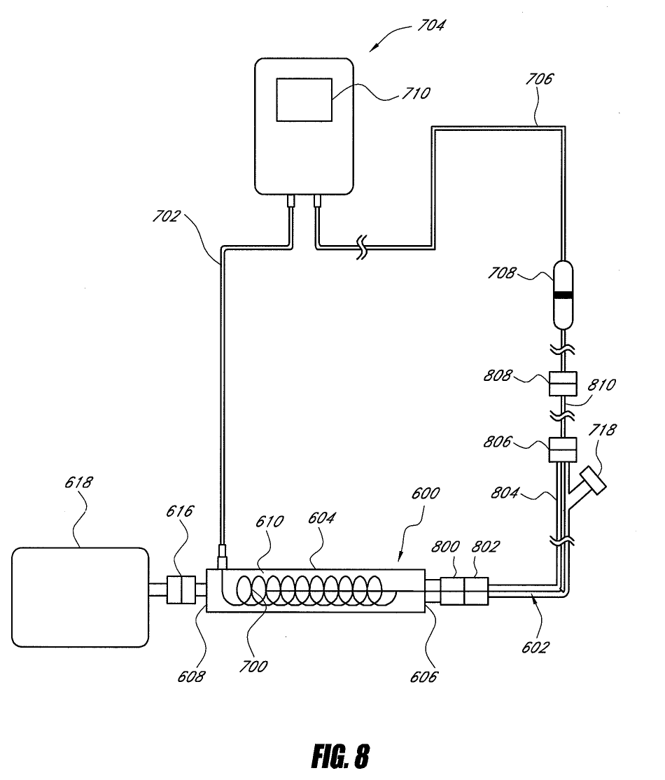

FIG. 8 shows another embodiment of a sensor calibration system 600 for calibrating a sensor 602, such as an arterial or central venous glucose sensor. The system 600 comprises a sensor 602 disposed in a sensor calibration chamber 604 with a proximal end 606 and a distal end 608 and a lumen 610 extending therethrough. The sensor 602 can comprise an elongate body with a distal portion comprising analyte sensing chemistry. In some embodiments, a valve 616, such as a one-way valve like, for example, a check valve, is attached to the distal end 608 of the sensor calibration chamber 604 and the other end of the valve 616 is attached to a bag 618 for receiving calibration solution. In some embodiments, the bag 618 encloses an absorption sponge 620 (not shown).

The calibration chamber 604 has a heater 700 for heating the calibration solution before calibration measurements are taken. The heater 700 can comprise a resistive heating element that is coiled around or within the calibration chamber 604. In some embodiments the heater 700 and heating element may be separate from the calibration chamber 604 and can be brought into contact with the calibration chamber 604 when heating of the calibration chamber 604 is required. In some embodiments, the heater 700 extends along a substantial portion of the calibration chamber 604, thereby facilitating rapid and uniform heating of the calibration fluid.

In some embodiments, the heater 700 can be powered via a power line 702 that can be connected to a glucose monitor 704, which can also be connected to the glucose sensor 602 via a glucose sensor line 706 and a glucose sensor connection interface 708. The glucose monitor 704 can have a display 710 for displaying instructions to the user for performing the calibration procedure. In addition, the display 710 can display the status of the calibration procedure, including the time to complete each step, the time remaining for each step, and the results of each step. For example, the display 710 can show the temperature of the calibration fluid and can show the results of each of the glucose measurements. Although the glucose monitor 704 and glucose sensor line 706 can be considered a part of the glucose calibration system 600, in some embodiments, the glucose monitor 704 and glucose sensor line 706 are separate from the glucose calibration system 600.

The proximal end 606 of the calibration chamber 604 can be attached to a connector 800 that matches the connectors used in an arterial line or central venous line. The glucose sensor 602 can have a corresponding connector 802 designed to be attached to an arterial line or central venous line connector. By using arterial line or central venous line connectors, the glucose sensor 602 can be seamlessly attached to both a calibration system 600 and then to an arterial line or central venous line after the glucose sensor 602 has been calibrated.

The corresponding connector 802 is attached to the distal end a protective sleeve 804. The proximal end of the protective sleeve can include both an infusion port 718 and a first valve 806, such as a Touhy-Borst valve. A second valve 808, such as a Touhy-Borst valve, can be placed proximally the first valve 806, with a slidable sheath 810 positioned therebetween. When both the first valve 806 and the second valve 808 are opened, the slidable sheath 810 can be inserted into the protective sleeve 804, thereby advancing the glucose sensor 602 into the calibration chamber 604. When calibration is completed, the slidable sheath 810 can be withdrawn from the protective sleeve 804, thereby withdrawing the glucose sensor 602 from the calibration chamber 604 and back into the protective sleeve 804. Insertion of the glucose sensor 604 through the arterial line or the central venous line and into the patient's vasculature can be accomplished in the same manner. The protective sleeve 804 provides protection to the glucose sensor 602 while the slidable sheath 810 allows clamping of the glucose sensor 602 by the first valve 806 and the second valve 808 on less sensitive portions of the glucose sensor 602.

To calibrate the glucose sensor 602, the connector 800 and the corresponding connector 802 of the glucose sensor 602 are connected together. Calibration solution with a known glucose concentration is introduced into the calibration chamber 604 via the infusion port 718 of the protective sleeve 804. For example, the first calibration solution can have a glucose concentration of 0 mg/dL. The glucose sensor 602 is introduced into the calibration chamber 604 via the connection between the connector 800 and corresponding connector 802. The glucose sensor 602 can be introduced into the calibration chamber 604 either before or after the calibration solution is introduced into the calibration chamber 604. The power line 702 and glucose sensor 602 are attached to the glucose monitor 704 and this step can be done either before or after the calibration fluid is introduced into the calibration chamber 604. The calibration solution is heated by the heater 700 to about the patient's body temperature, which generally is about 37 degrees Celsius. Once the calibration solution is heated to the target temperature, a first calibration measurement can be taken. If a second calibration measurement is desired, the first calibration solution can be drained and/or flushed into the bag 618 using, for example, a second calibration solution, which has a different glucose concentration than the first calibration solution. For example, the second calibration solution can have a glucose concentration of about 400 mg/dL. Sufficient second calibration solution can be used to flush the first solution to ensure that substantially all of the first calibration fluid is flushed into the bag 618. Once the second solution has replaced the first solution in the calibration chamber 604, the heater 700 can be used to heat the second solution to the patient's body temperature. Once the second solution is heated to the target temperature, a second calibration measurement can be taken. If additional calibration measurements are desired, for example a third calibration measurement, the steps of draining and/or flushing the previous calibration solution with the next calibration solution and then heating the next calibration solution before taking the calibration measurement can be repeated. For example, the third calibration solution can have a glucose concentration of about 100 mg/dL. In some embodiments, the calibration procedure can be shortened by calibrating first at 0 mg/dL, then at the highest level, e.g., 400 mg/dL, and then at an intermediate level, e.g., 100 mg/dL. This order can reduce calibration time where analyte detection involves reversible binding kinetics between the analyte and detector.

In some embodiments, the infusion port 718 can be switched with the one-way valve 616 and bag 618. In these embodiments, the infusion port 718 is attached to the calibration chamber 604 with or without an infusion line. The one-way valve 616 can be attached to proximal portion of the protective sleeve 804 and the bag 618 can be attached to the one-way valve.

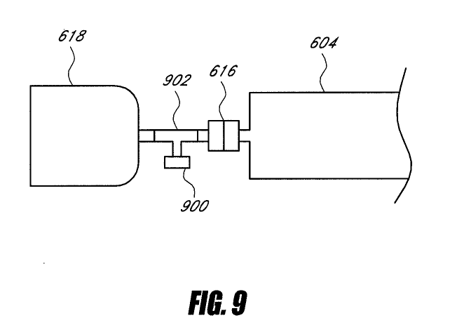

The embodiments described above, such as the embodiments shown in FIGS. 7 and 8, can be modified to include a vent to facilitate sterilization by, for example, ethylene oxide treatment. As illustrated in FIG. 9, a vent 900 can be located between the bag 618 and the one-way valve 616, which in some embodiments is attached to the calibration chamber 604. A three-way connector 902 can be used to join the bag 618 to both the one-way valve 616 and the vent 900. The vent 900 passes gasses such as ethylene oxide, but filters out microbial, particulate and liquid contaminants. This can be accomplished by incorporating, for example, a filter into the vent. The filter can have a pore size rated at less than or equal to about 0.22 μm or about 0.45 μm. In other embodiments, the vent 900 can be located at any other suitable location.

FIGS. 7 and 8 also show a schematic of a kit and two preferred embodiments of a calibration apparatus in accordance with the invention. Embodiments of the kits can include a glucose calibration system 600 comprising a glucose sensor 602, a calibration chamber 600 and a bag 618 as described above. In some embodiments, the glucose monitor 704, heater 700, and glucose sensor line 706 are reusable and are not part of the kit. In contrast, in some embodiments the kit components are disposable. The contents of the kits can be sterilized using, for example, ethylene oxide and can be supplied to the user in sterilized form. In addition, in the kit the glucose sensor 602 can be attached to the calibration chamber 604, and in some embodiments, the glucose sensor 602 can be inserted into the calibration chamber 604, so that calibration of the glucose sensor 602 can begin with the introduction of the first calibration solution into the calibration chamber 604.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

What is claimed is:

1. A method of calibrating an analyte sensor, the method comprising:
providing a vessel containing a first solution, wherein a sensing region of the sensor is in contact with said first solution;
obtaining a first calibration signal from the sensor;
adding an amount of a second solution into said vessel by means of a syringe, whereupon said sensor produces another calibration signal; and
calculating a calibration factor using said first calibration signal and any additional calibration signals, thereby calibrating the analyte sensor.

2. The method according to claim 1, further comprising:
repeating the step of adding an amount of a second solution into said vessel by means of a syringe, whereupon said sensor produces another calibration signal.

3. The method according to claim 2, wherein the step of adding an amount of a second solution into said vessel by means of a syringe, whereupon said sensor produces another calibration signal, is repeated twice.

4. The method according to claim 1, wherein said syringe has at least one stop for adding a premeasured amount of the second solution.

5. The method according to claim 1, wherein said analyte sensor is a glucose sensor.

6. The method according to claim 5, wherein said glucose sensor is an intravascular glucose sensor.

7. The method according to claim 5, wherein said second solution is a glucose solution.

8. The method according to claim 7, wherein said glucose solution has a concentration of glucose between 0 mg/dL and 10 g/dL.

9. The method according to claim 1, wherein said analyte sensor is a pH sensor.

10. The method according to claim 9, wherein said second solution is an acid.

11. The method according to claim 9, wherein said second solution is a base.

12. The method according to claim 1, wherein said vessel is a tonometer.

13. A kit for multipoint calibration of an analyte sensor comprising:
a vessel containing a calibration solution, wherein said vessel has a port; and
a syringe for delivery of an analyte.

14. The kit according to claim 13, wherein said vessel is a tonometer.

15. A method of calibrating an analyte sensor, the method comprising:
providing a vessel comprising at least two linearly adjacent chambers, wherein each chamber contains a solution, and wherein each chamber is separated from the chamber adjacent to it by a divider such that the solution in each chamber is substantially prevented from mixing with the solution in any other chamber; wherein a sensing region of the sensor is in contact with the solution in one of the chambers;
obtaining a first calibration signal from the sensor;
moving the sensing region of the sensor into an adjacent chamber, thereby contacting the sensing region with the solution in said adjacent chamber, whereupon the sensor produces an additional calibration signal; and
calculating a calibration factor using said first calibration signal and any additional calibration signals, thereby calibrating the analyte sensor.

16. The method according to claim 15, further comprising:
repeating the step of moving the sensing region of the sensor into an adjacent chamber, thereby contacting the sensing region with the solution in said adjacent chamber, whereupon the sensor produces a further additional calibration signal, until a calibration signal has been produced for each solution in each of the chambers.

17. The method according to claim 15, wherein said the step of moving the sensing region is carried out by retracting said sensor.

18. The method according to claim 15, wherein the step of moving the sensing region is carried out by advancing said sensor.

19. The method according to claim 15, wherein said sensor is a glucose sensor, and the solution in each chamber is a glucose solution.

20. The method according to claim 19, wherein said vessel comprises three linearly adjacent chambers: a first chamber, a middle chamber, and a last chamber.

21. The method according to claim 20, wherein said glucose solution in each chamber has a different concentration of glucose.

22. The method according to claim 21, wherein the glucose concentration of the solution increases from the first chamber to the last chamber.

23. The method according to claim 22, wherein the glucose concentration of the solution in the first chamber is 0 mg/dL, the glucose concentration of the solution in the middle chamber is 100 mg/dL, and glucose concentration of the solution in the last chamber is 400 mg/dL.

24. A method of calibrating an analyte sensor, the method comprising:
exposing the sensing region of the sensor to a solution, whereupon the sensor produces a first calibration signal;
combining at least one timed-release capsule with said solution, wherein said timed-release capsule contains an analyte;
allowing each timed-release capsule to release said analyte contained within it, whereupon the sensor produces another calibration signal; and
calculating a calibration factor using said first calibration signal and any additional calibration signals, thereby calibrating said analyte sensor.

25. The method according to claim 24, wherein said timed-release capsule takes between 10 seconds and 60 minutes to release said analyte contained within it.

26. The method according to claim 24, wherein said timed-release capsule comprises a degradable membrane.

27. The method according to claim 25, wherein said degradable membrane has a dissolution rate proportional to the thickness of said degradable membrane.

28. The method according to claim 24, wherein said method comprises combining three timed-release capsules with said solution.

29. The method according to claim 24, wherein said analyte sensor is a glucose sensor.

30. The method according to claim 29, wherein said analyte is glucose.

31. The method according to claim 30, wherein said glucose is in solution.

32. The method according to claim 30, wherein said glucose is not in solution.

33. The method according to claim 31, wherein said glucose has a concentration of between 0 mg/dL and 10 g/dL.

34. A method of calibrating an analyte sensor, the method comprising:
obtaining a vessel containing a solution, wherein a sensing region of the sensor is in contact with said solution; and wherein said vessel comprises at least one rupturable chamber containing an analyte, wherein said analyte is initially substantially separated from said solution;
obtaining a first calibration signal from the sensor;
rupturing each rupturable chamber, thereby releasing the analyte within it, whereupon the sensor produces another calibration signal; and
calculating a calibration factor using said first calibration signal and any additional calibration signals, thereby calibrating said analyte sensor.

35. The method according to claim 34, wherein said vessel comprises two rupturable chambers.

36. The method according to claim 34, wherein said analyte sensor is a glucose sensor.

37. The method according to claim 34, wherein said analyte is a glucose solution.

38. The method according to claim 37, wherein said glucose solution has a concentration of glucose between 0 mg/dL and 10 g/dL.

39. The method according to claim 34, wherein said rupturable chamber is rotatable, and wherein said rupturable chamber is ruptured by rotating said rupturable chamber, thereby releasing said analyte.

40. The method according to claim 39, wherein said rupturable chamber is ruptured by shearing when said rupturable chamber is rotated.

41. The method according to claim 39, wherein said rupturable chamber comprises a valve, wherein said valve remains in a closed position until said rupturable chamber is rotated, whereupon said valve opens, thereby releasing said analyte.

42. The method according to claim 34, wherein said rupturable chamber is ruptured by exerting pressure on said rupturable chamber, thereby rupturing said chamber and releasing said analyte.

43. A ready-to-calibrate and deploy, sterilized analyte sensor kit, comprising:
an analyte sensor comprising an elongate body having an indicator system disposed along a distal portion of the elongate body; and
a calibration vessel comprising a sensor port through which the distal portion of the sensor is sealably retained within the vessel until retracted for use, and the vessel further comprising a calibration means in fluid communication with the vessel,
wherein the sensor and vessel are pre-assembled, sterilized and sealed within a sterile package, ready for calibration and deployment.

44. The kit of claim 43, wherein the calibration means comprises a calibration port in fluid communication with the vessel and a syringe comprising a calibration solution fluidly-coupled to the vessel via the calibration port.

45. A ready-to-calibrate and deploy, sterilized analyte sensor kit, comprising:
an analyte sensor comprising an elongate body having an indicator system disposed along a distal portion of the elongate body and an coupling member configured to interface with an analyte monitor comprising a calibration algorithm;
a calibration apparatus comprising a calibration chamber sized to slidably receive and accommodate therein the distal portion of the elongate body of the sensor, an adjustable sealing means for sealing the distal portion within the calibration chamber, an infusion port fluidly coupled to the calibration chamber, and a fluid waste receptacle fluidly coupled to the calibration chamber; and
wherein the analyte sensor is slidably engaged within the calibration apparatus, sterilized and sealed within a sterile package, ready for calibration and deployment.

46. The kit of claim 45, further comprising a heater configured to heat the calibration chamber and a temperature sensor configured to measure the temperature within the calibration chamber.

47. A method of calibrating an analyte sensor, the method comprising:
providing the analyte sensor kit of claim 45;
providing at least first and second calibration solutions in separate syringes;
providing the analyte monitor;
coupling the analyte sensor to the analyte monitor via the coupling member;
initiating the calibration algorithm;
infusing the first calibration solution into the calibration chamber;
allowing the sensor to equilibrate;
infusing the second calibration solution into the calibration chamber, collecting displaced fluid in the waste receptacle; and
allowing the sensor to equilibrate, wherein the calibration algorithm automatically calibrates the sensor.

48. The method according to claim 47, further comprising:
providing a heater configured to heat the calibration chamber and a temperature sensor configured to measure the temperature within the calibration chamber;
heating the first calibration solution to a target temperature; and
heating the second calibration solution to the target temperature.
